(19) United States
(12) Patent Application Publication
(10) Pub. No.: US 2025/0389557 A1
(43) Pub. Date: Dec. 25, 2025

(54) MOISTURE METER WITH INTEGRATED PRINTER AND MEMORY

(71) Applicants: Anthony Quiles, Winchester, MA (US); Joshua Alan Goldstein, Melrose, MA (US)

(72) Inventors: Anthony Quiles, Winchester, MA (US); Joshua Alan Goldstein, Melrose, MA (US)

(21) Appl. No.: 19/064,261

(22) Filed: Feb. 26, 2025

Related U.S. Application Data
(60) Provisional application No. 63/557,647, filed on Feb. 26, 2024.

(51) Int. Cl. G01D 9/06; G01D 15/10; G01D 15/20; G01D 18/00; G01N 33/18; G01N 33/24; G08B 21/18; G08B 21/20

(52) U.S. Cl. CPC G01D 9/06; G01D 15/10; G01D 15/20; G01D 18/001; G01N 33/18; G01N 33/246; G08B 21/182; G08B 21/20

(57) ABSTRACT

Disclosed herein is a moisture meter comprising a housing, a moisture sensor, a processor configured to receive moisture readings from the moisture sensor, a display for displaying moisture data to a user, a printer integrated into the housing, and a power source for powering the moisture meter and printer.

### CROSS-REFERENCE TO RELATED APPLICATION

This application claims priority to and the benefit of U.S. Provisional Application No. 63/557,647 filed in the U.S. Patent and Trademark Office on Feb. 26, 2024. The foregoing patent application is incorporated herein by reference in its entirety for all purposes.

### SUMMARY

Disclosed herein are moisture meters with one or more of an integrated printing facility or a memory to enable longitudinal tracking of moisture data.

These and other systems, methods, objects, features, and advantages of the present disclosure will be apparent to those skilled in the art from the following detailed description of the preferred embodiment and the drawings.

All documents mentioned herein are hereby incorporated in their entirety by reference. References to items in the singular should be understood to include items in the plural, and vice versa, unless explicitly stated otherwise or clear from the text. Grammatical conjunctions are intended to express any and all disjunctive and conjunctive combinations of conjoined clauses, sentences, words, and the like, unless otherwise stated or clear from the context.

### BRIEF DESCRIPTION OF THE FIGURES

The disclosure and the following detailed description of certain embodiments thereof may be understood by reference to the following figures:

FIG. 1 depicts an embodiment of a moisture meter of the disclosure.

FIG. 2 depicts various user interfaces for one or more applications operable with the moisture meter of the disclosure.

FIG. 3 depicts an embodiment of a moisture meter of the disclosure having an integrated printing facility with a current printout, and with: A) the connected moisture sensor uncovered; and B) the connected moisture sensor covered.

FIGS. 4A and 4B depict the operational interface for the pin-type moisture sensor.

FIG. 5A depicts the moisture sensor in operational connection to the interface depicted in FIGS. 4A and 4B.

FIG. 5B depicts an embodiment of a moisture meter of the disclosure.

FIG. 6 depicts various views of the moisture meter housing: A) back side of the housing with a view of the power source compartment; B) left side of the housing depicting an optional input for power; C) right side of the housing depicting the connection to the remote, pin-type sensor.

FIG. 7A depicts the moisture meter collecting control data and FIG. 7B depicts the moisture meter collecting experimental data.

FIG. 8A depicts an embodiment of a moisture meter of the disclosure.

FIG. 8B depicts a printout from the integrated printing facility of the moisture meter.

### DETAILED DESCRIPTION

Moisture meters disclosed herein may include one or more of an integrated printing facility or a memory to enable longitudinal tracking of moisture data. Integrating printing enables on-demand provision of data, optionally at the moment of capture, including moisture data, temperature data, location data, date/time data, and other notes or markings. While examples throughout this disclosure depict the moisture meter with an integrated facility, it should be understood that the moisture meter may optionally have a communications facility, such as Bluetooth or WiFi, that can transmit data for printing to a remote printer or for processing/storage at a remote computing facility (e.g., smartphone, computer, the cloud, server, etc.).

Also disclosed herein are moisture meters with location sensing facilities or location tracking facilities to enable moisture data tracking in coordination with location. An integrated memory facilitates moisture mapping by storing moisture data optionally in coordination with a floor plan of the facility being evaluated for moisture. Further disclosed herein are moisture meters that enable validated, tamper-proof data for sharing with agencies and other purposes, such as to provide a certified moisture reading. Also disclosed herein are moisture meters with communication facilities enabling wireless transmission of data and receipt of firmware upgrades and operating instructions. Example embodiments throughout this disclosure depict the moisture meter with a remote, connected moisture sensor connected to the housing by a cord in electrical communication with the processor/display, but it should be understood that the moisture sensor may also be integrated with the housing.

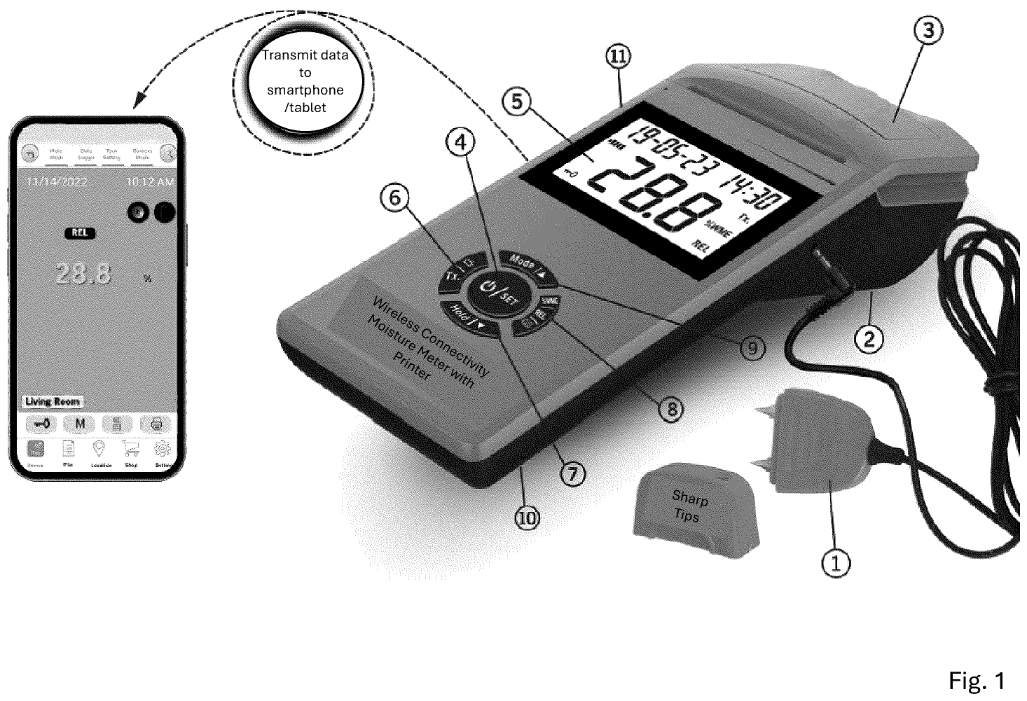

Referring to FIG. 1, a moisture meter is depicted with a remote pin-type sensor 1 and a non-destructive type sensor up to 2″ depth. While the examples herein demonstrate a moisture meter with physical buttons, it should be understood that the moisture meter may have a touch screen or other electronic user interface. The moisture meter may have an integrated label printer 3, as known in the art, with programmable fields for printing, such as room, date/time, units of measurement, location, temperature, room name, tracking ID, number of measurement in a dataset, and the like. The moisture meter includes a power/set button 4, an LCD display and support backlight 5, and a button to turn on/off backlight and or transmission facilities 6 depending on if actuated persistently or once. In some embodiments, functions assigned to a single button that are dependent on the length of press of the button may be separated into two buttons. In embodiments, the moisture meter may include a hold/freeze button 7 that can be actuated to hold or release the reading, such as if pressed persistently, or may have a different function when pressed once, such as to decrease the value in setting mode. The moisture meter may have a % WME/REL Measuring Mode and Print button 8. In one mode, the button may be pressed persistently to print data, such as frozen data. In another mode, the button may be pressed once to switch between % WME (e.g., pin type measurement mode) and REL (pinless type measurement mode). The moisture meter may include a mode/up button 9 which may be pressed persistently to enter date/time setting mode or once to increase the value in the setting mode. The moisture meter may include a rechargeable lithium battery 10 (e.g. 7.4 V, 1500 mA) and/or USB Type C charging 11.

In embodiments, the display may present current or historical moisture readings, including metadata and associated sensed data such as date, time, room number, location, temperature, atmospheric pressure, light levels, and the like. In some embodiments, the display may present the moisture readings on a floor plan. For example, an integrated GPS may associate a location with the sensed moisture and then associate the geo-located moisture data with a position on a floor plan or other map. In some embodiments the display may present data for printing and/or transmission, such as the floor plan with geo-located moisture data. In embodiments, the display may be color or monochromatic.

In embodiments, the housing may have an integrated light for illuminating a workspace. In embodiments, the housing may have an integrated speaker for outputting data in an audible manner. In embodiments, the housing may have a level and/or laser beam pointer.

In embodiments, the moisture meter includes a housing and a moisture sensor, wherein the moisture sensor is remote or integrated. The moisture meter may further include a processor configured to receive moisture readings from the moisture sensor, which may be integrated in the housing, remotely connected, or in electronic communication with the moisture sensor. The moisture meter may include a display for displaying moisture data to a user. The moisture meter may include a printer integrated into the housing, such as a thermal printer. The printer is configured to print moisture data onto a paper roll, onto adhesive labels, or the like. The moisture meter may include a power source for powering the moisture meter and printer. The moisture meter may include a memory module configured to store moisture readings and print logs. The moisture meter may include a communication module or facility configured to transfer moisture data to an external device or repository, wherein the communication module is configured to transmit moisture data wirelessly (e.g., Bluetooth, WiFi, cellular, satellite, etc.). The moisture meter may include a calibration module configured to calibrate the moisture sensor. The moisture meter may include a warning module configured to alert a user when moisture readings exceed a predetermined threshold.

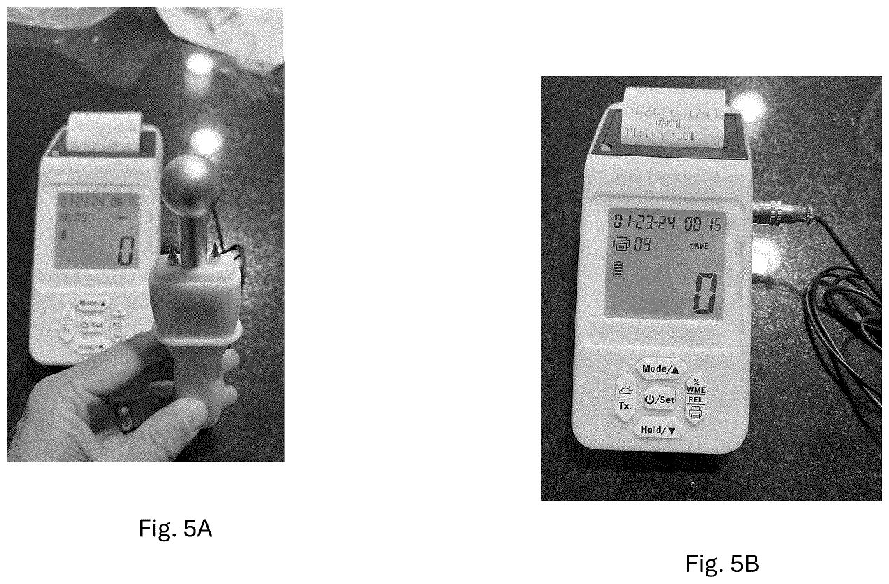

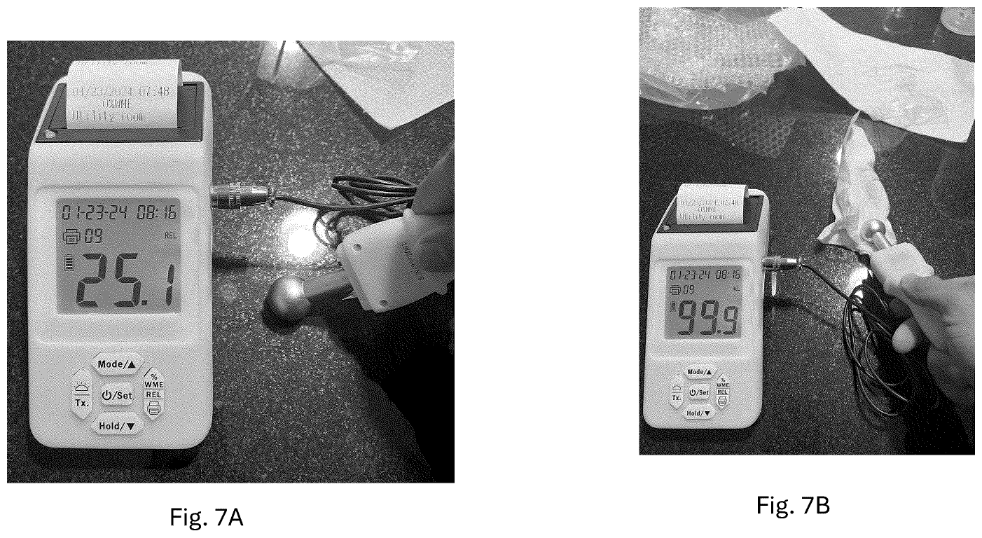

In embodiments, a method of measuring and printing moisture data may include obtaining moisture readings from a surface, processing the moisture readings to generate moisture data, displaying the moisture data on the moisture meter, and printing the moisture data onto a medium using the integrated printer. As shown in FIG. 5A, the moisture sensor may be exposed in preparation for use and, as in FIG. 7B, placed adjacent to an item to be evaluated for moisture. Alternatively, ambient moisture may be evaluated with the moisture meter.

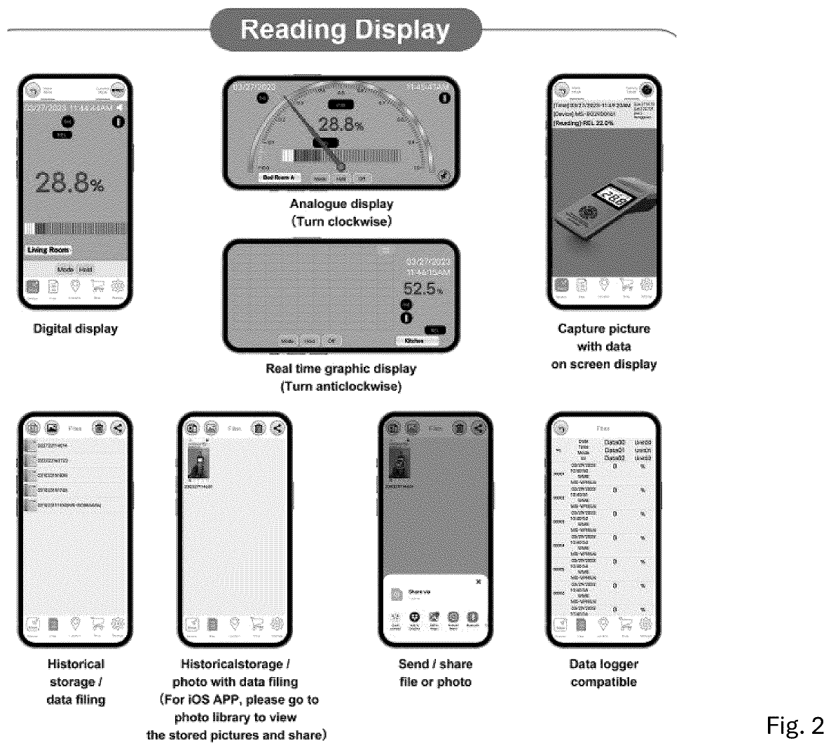

FIG. 2 depicts various user interfaces for one or more applications operable with the moisture meter of the disclosure, including a digital display, analogue display (such as when a smartphone executing the application is turned clockwise), a real time graphic display (such as when a smartphone executing the application is turned anti-clockwise), a capture picture with data on screen display, a historical storage/data filing display, a historical storage/photo with data filing display, a send/share file or photo display, a data logger compatible display, and the like. In other embodiments, the application may display a floor plan and moisture readings may be displayed on the floor plan in accordance with where the moisture reading was taken.

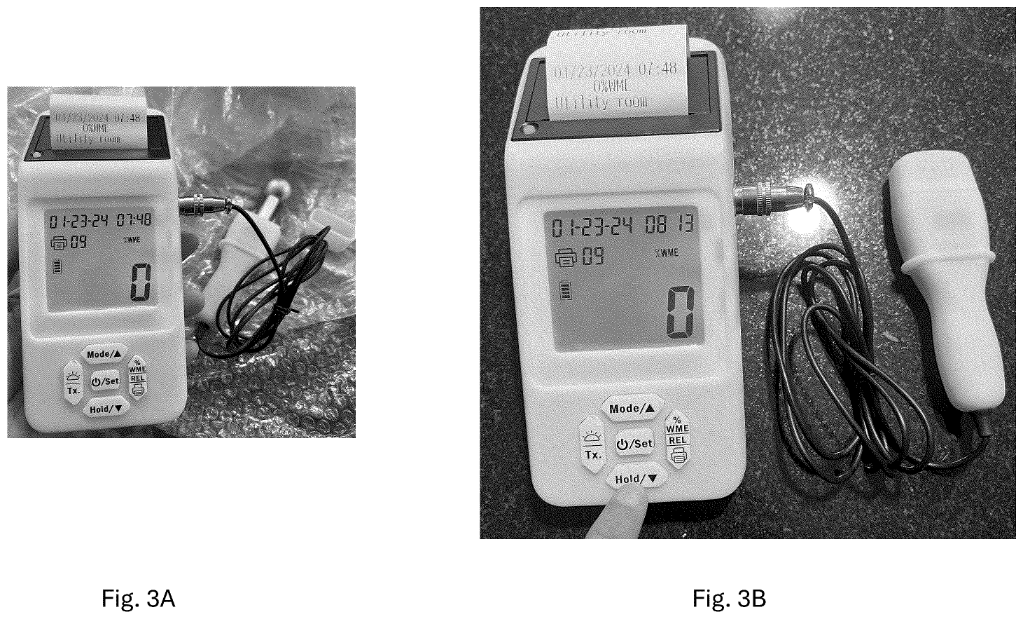

FIG. 3 depicts an embodiment of a moisture meter of the disclosure having an integrated printing facility with a current printout, and with: A) the connected moisture sensor uncovered; and B) the connected moisture sensor covered.

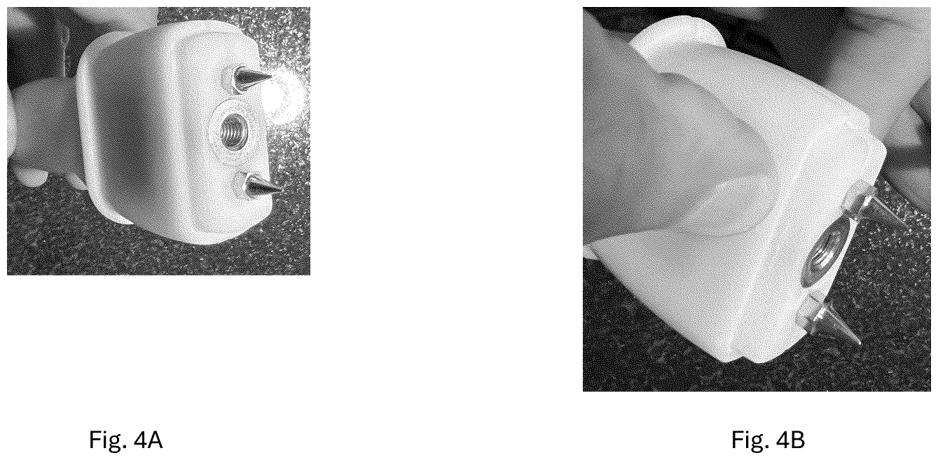

FIGS. 4A and 4B depict the operational interface for the pin-type moisture sensor.

FIG. 5A depicts the moisture sensor in operational connection to the interface depicted in FIGS. 4A and 4B.

FIG. 5B depicts an embodiment of a moisture meter of the disclosure.

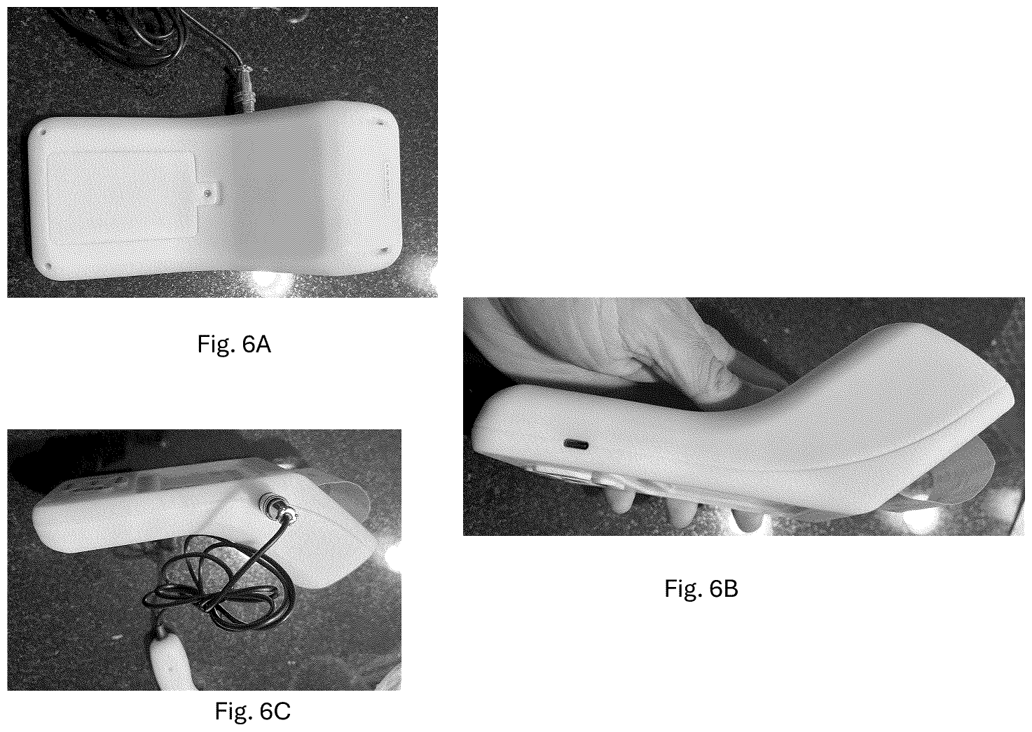

FIG. 6 depicts various views of the moisture meter housing: A) back side of the housing with a view of the power source compartment; B) left side of the housing depicting an optional input for power; C) right side of the housing depicting the connection to the remote, pin-type sensor.

FIG. 7A depicts the moisture meter collecting control data and FIG. 7B depicts the moisture meter collecting experimental data.

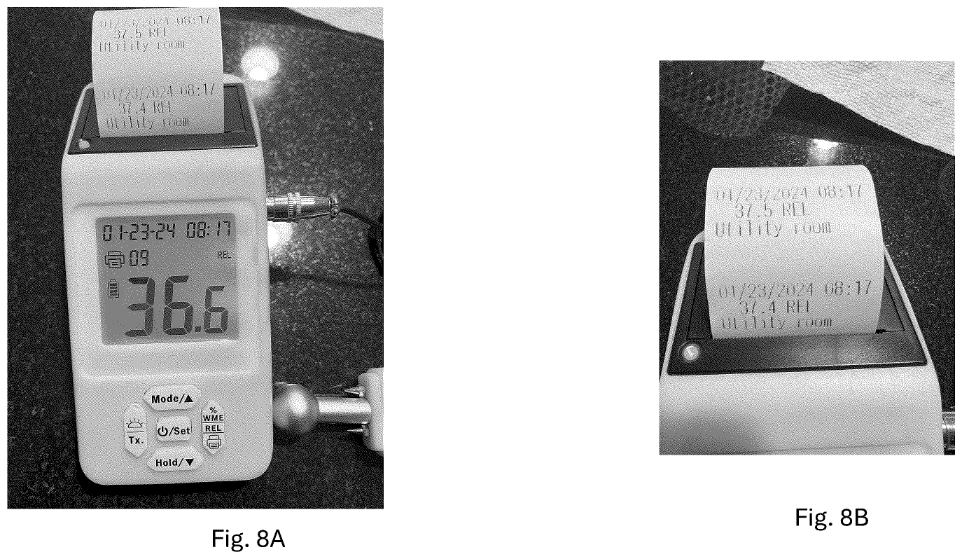

FIG. 8A depicts an embodiment of a moisture meter of the disclosure.

FIG. 8B depicts a printout from the integrated printing facility of the moisture meter. Some printouts may include a floor plan and moisture readings may be displayed on the floor plan in accordance with where the moisture reading was taken.

The methods and systems described herein may be deployed in part or in whole through a machine having a computer, computing device, processor, circuit, and/or server that executes computer readable instructions, program codes, instructions, and/or includes hardware configured to functionally execute one or more operations of the methods and systems disclosed herein. The terms computer, computing device, processor, circuit, and/or server, as utilized herein, should be understood broadly.

Any one or more of the terms computer, computing device, processor, circuit, and/or server include a computer of any type, capable to access instructions stored in communication thereto such as upon a non-transient computer readable medium, whereupon the computer performs operations of systems or methods described herein upon executing the instructions. In certain embodiments, such instructions themselves comprise a computer, computing device, processor, circuit, and/or server. Additionally or alternatively, a computer, computing device, processor, circuit, and/or server may be a separate hardware device, one or more computing resources distributed across hardware devices, and/or may include such aspects as logical circuits, embedded circuits, sensors, actuators, input and/or output devices, network and/or communication resources, memory resources of any type, processing resources of any type, and/or hardware devices configured to be responsive to determined conditions to functionally execute one or more operations of systems and methods herein.

Network and/or communication resources include, without limitation, local area network, wide area network, wireless, internet, or any other known communication resources and protocols. Example and non-limiting hardware, computers, computing devices, processors, circuits, and/or servers include, without limitation, a general purpose computer, a server, an embedded computer, a mobile device, a virtual machine, and/or an emulated version of one or more of these. Example and non-limiting hardware, computers, computing devices, processors, circuits, and/or servers may be physical, logical, or virtual.

Thus, in one aspect, each method described above and combinations thereof may be embodied in computer executable code that, when executing on one or more computing devices, performs the steps thereof. In another aspect, the methods may be embodied in systems that perform the steps thereof, and may be distributed across devices in a number of ways, or all of the functionality may be integrated into a dedicated, standalone device or other hardware. In another aspect, the means for performing the steps associated with the processes described above may include any of the hardware and/or computer-readable instructions described above. All such permutations and combinations are contemplated in embodiments of the present disclosure.

While the disclosure has been disclosed in connection with the preferred embodiments shown and described in detail, various modifications and improvements thereon will become readily apparent to those skilled in the art. Accordingly, the spirit and scope of the present disclosure is not to be limited by the foregoing examples, but is to be understood in the broadest sense allowable by law.

### ADDITIONAL STATEMENTS OF THE DISCLOSURE

In some implementations, the disclosed moisture meter may be described in the following numbered clauses or otherwise described herein and as illustrated in FIGS. 1-8. The disclosure includes the following clauses:

1. A moisture meter, comprising: a) a housing; b) a processor configured to receive moisture readings from a moisture sensor; c) a display for displaying moisture data to a user; and d) a printer integrated into the housing configured to receive information from the processor to print.

2. The moisture meter of clause 1, further comprising, a power source for powering the moisture meter.

3. The moisture meter of clause 1, wherein the moisture sensor is disposed within the housing.

4. The moisture meter of clause 1, wherein the moisture sensor is in remote communication with the moisture meter.

5. The moisture meter of clause 1, wherein the printer is a thermal printer.

6. The moisture meter of clause 1, wherein the printer is configured to print moisture data onto a paper roll.

7. The moisture meter of clause 1, wherein the printer is configured to print moisture data onto adhesive labels.

8. The moisture meter of clause 1, further comprising a memory module configured to store moisture readings and print logs.

9. The moisture meter of clause 1, further comprising a communications facility within the housing configured to transmit the moisture data to an external device.

10. The moisture meter of clause 9, wherein the communications facility is configured to transmit moisture data wirelessly.

11. The moisture meter of clause 1, further comprising a calibration module configured to calibrate the moisture sensor.

12. The moisture meter of clause 1, further comprising a warning module configured to alert the user when moisture readings exceed a predetermined threshold.

13. The moisture meter of clause 1, further comprising a user interface with a touch screen display on the housing.

14. The moisture meter of clause 1, wherein the housing further comprises an integrated light for illuminating a workspace.

15. The moisture meter of clause 9, wherein the processor is further configured to receive firmware upgrades wirelessly through the communications facility.

16. The moisture meter of clause 1, wherein the moisture sensor is at least one of a pin-type sensor or a non-destructive type sensor.

17. The moisture meter of clause 1, wherein the moisture sensor is connected to the housing by a cord.

18. The moisture meter of clause 1, wherein the housing further comprises a level and/or laser beam pointer.

19. A moisture meter, comprising: a) a housing; b) a moisture sensor; c) a processor configured to receive moisture readings from the moisture sensor; d) a display for displaying moisture data to a user, the moisture data including the moisture readings; and e) a memory for storing the moisture data.

20. A moisture meter, comprising: a) a housing; b) a moisture sensor; c) a processor configured to receive moisture readings from the moisture sensor; d) a display for displaying moisture data to a user, the moisture data including the moisture readings; and e) a communications facility within the housing configured to transmit the moisture data to an external device.

21. A method of measuring and printing moisture data comprising: a) obtaining moisture readings from a surface using a moisture meter; b) processing the moisture readings to generate moisture data; and c) printing the moisture data onto a medium using an integrated printer of the moisture meter.

22. The method of clause 21, further comprising, displaying the moisture data on a floor plan, wherein location data associated with the moisture data is obtained from an integrated GPS receiver within a housing of the moisture meter.

What is claimed is:

1. A moisture meter, comprising:
a) a housing;
b) a processor configured to receive moisture readings from a moisture sensor;
c) a display for displaying moisture data to a user; and
d) a printer integrated into the housing configured to receive information from the processor to print.

2. The moisture meter of claim 1, further comprising, a power source for powering the moisture meter.

3. The moisture meter of claim 1, wherein the moisture sensor is disposed within the housing.

4. The moisture meter of claim 1, wherein the moisture sensor is in remote communication with the moisture meter.

5. The moisture meter of claim 1, wherein the printer is a thermal printer.

6. The moisture meter of claim 1, wherein the printer is configured to print moisture data onto a paper roll.

7. The moisture meter of claim 1, wherein the printer is configured to print moisture data onto adhesive labels.

8. The moisture meter of claim 1, further comprising a memory configured to store moisture readings and print logs.

9. The moisture meter of claim 1, further comprising a communications facility within the housing configured to transmit the moisture data to an external device.

10. The moisture meter of claim 1, further comprising a calibration module configured to calibrate the moisture sensor.

11. The moisture meter of claim 1, further comprising a warning module configured to alert the user when moisture readings exceed a predetermined threshold.

12. The moisture meter of claim 1, further comprising a user interface with a touch screen display on the housing.

13. The moisture meter of claim 1, wherein the moisture sensor is at least one of a pin-type sensor or a non-destructive type sensor.

14. The moisture meter of claim 1, wherein the moisture sensor is connected to the housing by a cord.

15. A moisture meter, comprising:
a) a housing;
b) a moisture sensor;
c) a processor configured to receive moisture readings from the moisture sensor;
d) a display for displaying moisture data to a user, the moisture data including the moisture readings; and
e) a memory for storing the moisture data.

16. The moisture meter of claim 15, further comprising a communications facility within the housing configured to transmit the moisture data to an external device.

17. A method of measuring and printing moisture data comprising:
a) obtaining moisture readings from a surface using a moisture meter;
b) processing the moisture readings to generate moisture data; and
c) printing the moisture data onto a medium using an integrated printer of the moisture meter.

18. The method of claim 17, further comprising, associating the moisture data with location data to obtain geo-located moisture data, wherein location data is obtained from an integrated GPS receiver within a housing of the moisture meter.

19. The method of claim 18, further comprising, displaying the geo-located moisture data on a floor plan.

20. The method of claim 18, further comprising, printing a floor plan comprising the geo-located moisture data.
